(19) United States
(12) Patent Application Publication
(10) Pub. No.: US 2006/0024810 A1
(43) Pub. Date: Feb. 2, 2006

(54) Method of atttaching nanotubes to bacteria and applications

(76) Inventors: Surendra Bandopant Khadkikar, Pune (IN); Erach Aspandiar Irani, Mumbai (IN)

Correspondence Address: ERACH A. IRANI;C/O TOY KINGDOM, 106 B. DESAI ROAD, MUMBAI, 400036, IN

(21) Appl. No.: 10/899,500

(22) Filed: Jul. 27, 2004

(51) Int. Cl. C12N 1/20; C12N 15/74

(52) U.S. Cl. 435/252.1; 435/471

(57) ABSTRACT

A method of attaching nano-tubes to unicellular organisms such as bacteria and plankton is proposed. The method should work for other loosely multi-cellular organisms such as some species of fungii. After attaching these nano-tubes two types of applications are specifically presented. The first type of application relies on the individual properties of bacteria with nano-tubes attached to them. In this kind of application, we discuss cancer cure that is applicable for removing all solid tumours in the human and other animal body. The second kind of application relies on the collective properties of bacteria. In this kind of application, we discuss the induction of collective identity in bacteria to promote bio-intelligence in bacteria.

### REFERENCES CITED

US Patent Documents

| | | |
|---|---|---|
| 6,763,338 | 4/2002 Kirshenbaum; Evan R | 706/12 |
| 5,343,554 | 8/1994 Koza, et. al. | 706/13 |
| 6,752,994 | 6/2004 Jacobs, Jr. et al. | 424/248.1 |
| 6,762,331 | 7/2004 Hong, et. al. | 568/732 |
| 5,581,091 | 12/1996 Moskovits, et. al. | 257/9 |
| 6,763,341 | 7/2004 Okude | 706/5 |
| 6,763,354 | 7/2004 Hosken | 707/6 |
| 6,424,961 | 7/2002 Ayala | 706/25 |

1. Mills, D. R., Peterson, R. L., and Spiegelman, S. An Extracellular Darwinian Experiment with a Self-Duplicating Nucleic Acid Molecule. Proc. Natl. Acad. Sci. USA 58: 217-224., 1967
2. Lenski, R. E., and Travisano, M. Dynamics of Adaptation and Diversification: A 10,000-Generation Experiment with Bacterial Populations. Proc. Natl. Acad. Sci. USA 91:6808-6814, 1994.
3. Elena, S.F., Cooper, V. S., and Lenski, R. E. Punctuated Evolution Caused By Selection of Rare Beneficial Mutations. Science 272: 1802-1804, 1996.
4. Dobzhansky, T., and Pavlovsky, O., 1971. Experimentally Created Incipient Species of Drosophila. Nature 230: 289-292
5. Stuart J. Russell, Peter Norvig, “Artificial Intelligence: A Modem Approach (2^{nd} Edition)”, Prentice Hall, 2nd edition (December 2002).
6. Thomas Back, “Evolutionary Algorithms in Theory and Practise: Evolution Strategies, Evolutionary Programming, Genetic Algorithms”, Oxford University Press, January 1996. ISBN: 0195099710.
7. Skapura, David M., “Building Neural Networks”. Menlo Park, Calif.: Addison-Wesley Publishing Company, 1996.
8. David E. Goldburg, “Genetic Algorithms in Search, Optimization and Machine Learning”, Addison-Wesley Professional, January 1989. ISBN 0201157675.
9. E. Bonabeau and G. Theraulaz, “Swarm smarts”, Scientific American, pp. 72-79, March 2000
10. Malik, O. 2002. Distributed Computing Grid Networks: New Grid Networks Put Idle Computing Power to Work. Red Herring October 2002: 39-41
11. Lee H., Purdon A. M., Chu V, Westervelt R. M. “Controlled Assembly of Magnetic Nanoparticles from Magnetotactic Bacteria using Microelectromagnets Arrays”, Nano Letters, May 2004, Vol. 4, Issue 5, pg 995.
12. Bahaj, A. S., James P. A. B., Ellwood D. C., Watson J. H. P., “Characterization and growth of magnetotactic bacteria: Implications of clean up of environmental pollution”, Journal of Applied Physics, May 1993, Vol. 73, Issue 10, pg. 5394.
13. Paul L. McEuen, “Carbon-based Electronics”, Nature 393, 15 (1998).

### FIELD OF INVENTION

The present invention is directed to provide a method for attaching nano-tubes to unicellular organisms such as bacteria and plankton and loosely multi-cellular organisms such as fungi. These so modified unicellular organisms are sought to be used for curing cancer in animals, specifically humans, by exploiting their properties as individual organisms. These unicellular organisms, whether so modified to have nano-tubes attached to them individually, are also sought to be used for collective properties and to be trained. These collectively trained organisms are then sought to be trained and educated using the formative principles of artificial intelligence.

### DESCRIPTION OF THE BACKGROUND ACTIVITY OF THE ART

Carbon and other nanotubes are being investigated for several applications. The cost of carbon nanotubes is dropping making it feasible to use carbon nanotubes in several applications. Microbiology and genetics as scientific fields have advanced so much that it is possible to sequence the genomes of any plant or animal organism or unicellular organism or multi-cellular organism or virus fairly quickly and easily.

Artificial Intelligence [5] as a field has made significant advances since LISP was invented and symbolic mathematic integration has been used. The science and art of computer programming has also significantly developed. The use of compilers and advanced programming languages such as C++ and visual development environments such as those used commercially for Visual C++ and Visual Basic has also significantly developed. Within artificial intelligence the field that is rapidly maturing is evolutionary intelligence [6] including neural networks [7] and genetic programming [8]. Swarm intelligence [9] is another topic of study. These modes of artificial intelligence seek to emulate nature in some respects on a silicon-based conventional digital computer and may use a super-computer or a grid computer[10].

Organisms in nature with fairly complex genetic codes have the unique property that they are extremely rapid in changing their genetic codes in response to the environment or to specific attempts to mutate them in a particular direction or what we can term as “guided mutation”. The bacteriophage virus Q-beta[1] responds extremely rapidly in terms of number of generations to mutations designed to guide it to reduce its genome length substantially in a few dozen generations. The E. coli virus [2] responds similarly rapidly to a new nutrient environment. The Drosophila fruit fly responds with a new species when researchers induced it to do so in a few hundred generations [4].

Current wisdom holds that nanotubes pass through bacteria, killing them. However, the magnetotactic bacteria [11, 12] incorporate ferrous nanotubes in them.

### OBJECTS OF THE PRESENT INVENTION

The present invention aims to define a process by which bacteria can be mutated until they survive carbon (or other) nano-tubes. This process of mutation is extended until the bacteria develop a collective identity that simulates latent thinking. These latent thinking bacteria are further mutated until the latent thinking becomes to resemble an education. The object of this invention is to produce these bacteria mutated with nanotubes until they learn to survive nanotubes, learn to use nanotubes as tools, and learn to have collective properties of latent thinking because they have to survive nanotubes. Examples of use of the bacteria showing individual and collective properties are given.

The individual properties of the bacteria are used to remove cancer tumors from the body.

The collective thinking bacteria are sought to be trained and educated until they show biological artificial intelligence.

### SUMMARY OF THE INVENTION

We hypothesize that it should be possible to mutate bacteria (or other unicellular organisms such as plankton or loosely multicellular organisms such as fungi) to make them either attach carbon or other nanotubes on the surface of their cell-walls or to incorporate them within their cell walls. These mutations can be accomplished by giving the bacteria a plentiful supply of agar mixed with nano-tubes or nano-structures until the bacteria mutate to attach nanotubes on their surface or within their cell wall. If necessary, the magnetotactic bacteria can be added to the researcher's container where the bacteria are being mutated, so that the bacteria being mutated learn to incorporate carbon or other nanotubes within their cell-walls or on their cell-walls.

This use of guided mutation has so far not been reported in the literature since it is the bringing together of three advanced scientific fields, viz. nanotechnology, artificial intelligence, and -genetic manipulation of bacteria and/or other unicellular or loosely multicellular organisms.

### DESCRIPTION OF THE DRAWINGS

No drawings are provided.

### DETAILED DESCRIPTIONS

Sub-Process 1 (SP1): Guided Mutation of Bacteria to Survive Carbon Nanotubes

In this first process we mutate bacteria (or other unicellular organisms such as plankton or loosely multi-cellular organisms such as fungi) to make them either attach carbon or other nanotubes on the surface of their cell-walls or to incorporate them within their cell walls. These mutations can be accomplished by giving the bacteria a plentiful supply of agar mixed with nanotubes or nano-structures until the bacteria mutate to attach nanotubes on their surface or within their cell wall. If necessary, the magnetotactic bacteria can be added to the researcher's container where the bacteria are being mutated, so that the bacteria being mutated learn to incorporate carbon or other nanotubes within their cell-walls or on their cell-walls. Carbon nanotubes are specifically chosen because they have electronic properties that can be useful much later when the bacteria might want to “think” at MegaHertz and GigaHertz speeds [13].

Sub-Process 2 (SP2): Making Bacteria from SP1 Use Nano-Tubes as Tools

Once the bacteria (or other mutated organisms) learn to live with nanotubes (carbon or non-carbon) they will start using these nano-tubes to beneficial purposes for themselves, perhaps to fight with other bacteria in the hunt for food or for play. The wealth of these carbon nano-tubes has to be lost if the bacteria go into a cyst escaping stressful conditions. Thus there should arise mutations of bacteria that will not go into a cyst so easily but try to retain their wealth of carbon nano-tubes.

Sub-Process 3 (SP3): Forming a Collective of Bacteria in Spherical Balls, but Not Yet Thinking

We further hypothesize that if the bacteria that have mutated to use carbon nano-tubes as tools are exposed to mechanical stressors such as fullerones thrown at them (fullerones are semi-spherical carbon nano-structures), the bacteria will learn to form a sphere with the bacteria on the surface of the sphere being those ones that have adapted to incorporate carbon nanotubes in them or somehow mutated to deflect the fullerones being thrown at them while the inner part of the sphere will consist of relatively soft bacteria. These bacteria will learn to be in symbiosis with each other merely to continue. their survival. Thus a collective symbiosis will be forced on the unicellular bacteria.

Sub-Process 4 (SP4): Making the Spherical-Ball Collective of Bacteria THINK

Once the bacteria forms a collective symbiosis, we stress the bacterial spheres so formed with additional stressors such as a higher intensity of nano-structures, fullerones, and nano-tubes being thrown at the sphere of bacteria, radio waves, mechanical agitation, magnetic field variations and so on. As the bacteria resist these impulses to make the spherical ball break-up, they learn to communicate amongst each other, and co-operate amongst each other. An unconscious notion of a collective identity is formed among the originally unicellular bacteria. This is the beginning of latent “thinking” as a collective consciousness in the bacteria.

Process 5 (SP5): Enriching the Language of the Bacteria Collective Spheres (Balls) Both Individually Within a Collective Sphere (Ball) and Across Collective Spheres (Balls)

Once the bacteria for collective conglomerates that are thinking (as in Sub Process SP4) we enrich the language used inside the collective conglomerate through external processes. Language is the ability to convert experience into abstract symbols and it is a means of communication within a living entity (collective sphere) or from one entity (collective sphere) to another living entity (collective sphere). Several collective spheres (or balls) will acquire the same symbolism or language for the same experience and communicate with each other.

We can use exposure to different chemical or bio-chemical signals, different electrical stimulation, different magnetic stimulation, different electromagnetic stimulation, acoustic stimulation, mechanical stimulation, forcing in contact with other collective bodies, living cells, dead cells, cells in-vitro and in-vivo to stimulate the bacterial collectives.

In the above fashion, we propose to impress on bacteria a thinking collective that can be used to produce a self-programming computer with biological artificial intelligence. This exploits the collective properties of the bacteria and their ability to have language that living things innately have.

Exploiting Bacteria's Individual Property With Nano-Tubes

In order to exploit the individual property of the bacteria with nano-tubes we select those bacteria that are present in particular portions of the animal body (including human body) and in-vitro mutate them to incorporate nano-tubes in them by mixing nano-tubes, agar and agitating the mixture. The process outlined in Sub-Process 1 (SP1): Guided mutation of bacteria to survive carbon nanotubes is used.

These bacteria are then injected with agar into the part of the body where the cancer lump is present. The immune system attacks the bacteria forcing them further into the lump. Since the carbon nano-tubes are hard the bacteria can dig into the lump and break the lump into small portions that can be removed by the body. When the lump is removed, antibiotics for that specific strain of bacteria are administered and the bacteria are killed.

We claim:

1. A process of preparing bacteria adapted to using carbon nano-tubes as tools by starting with raw bacteria and putting them in a vessel and putting agar (food) with nano-tubes in the same vessel and agitating the mixture thus propelling the nano-tubes to penetrate the cell walls of the bacteria until they mutate to develop a defence against them.

2. A process of mutating the bacteria from the bacteria in claim 1 by exposing them to increased concentrations of nanotubes in agar, until the bacteria form approximately spherically shaped balls, which are exposed to further increased concentrations of nanotubes and other nano-structures in agar until the bacteria mutate for their collective survival to communicate and co-operate with each other and so collectively think.

3. A process of taking the bacteria from the human body and mutating them as in claim one till they incorporate carbon nanotubes on their cell walls. The bacteria are then injected or inserted with agar into the human body where cancer lumps are present so that they are either injected into the cancer lump or bathe the cancer lump. These bacteria then attack the cancer lump breaking into the lump and fragmenting the lump. These fragments can then removed by the body. When the lump is completely removed, antibiotics for that strain of bacteria are administered and the cancer is removed from the body.

4. The process of claim 1 using nanotubes of any kind other than carbon.

5. The process of claim 1 using nano-structures besides carbon nano-tubes.

6. The process of claim 3 using nano-structures other than carbon nanotubes.

7. The process of claim 2 where the bacteria may form into collective structures other than spherical balls.

8. The process of claim 3 using bacteria other than bacteria from the human body but from any animal body of any animal species and injecting the bacteria after adapting them to carbon nano-tubes in-vitro into animals of the same species.

9. The process of claim 1 where besides physically agitating the mixture, the mixture is agitated using radiowaves, magnetic fields.

10. The process of claim 3 where the bacteria of claim 1 are injected into the cancerous lump but without agar.

11. Bacteria formed into collective spheres from the process of claim 2 and exposed to mechanical stimuli, acoustic stimuli, chemical stimuli, bio-chemical stimuli, electromagnetic stimuli of short or long-wavelengths, or bio-chemical stimuli, or nanotubes and signals in the above stimuli. These will result in enrichment of the bacteria's language ability within collective spheres and across collective spheres.

12. Bacteria formed into collective spheres from the process of claim 2 brought into contact with same or other species of bacteria formed into collective spheres from the process of claim 2.

13. Bacteria formed into collective spheres from the process of claim 2 brought into contact with different bacteria that maybe of the same species or different but that have formed collectives using different types of nanotubes.

14. Bacteria formed into collective spheres exposed to cells that are cancerous.

15. Exposure to the collectively balled bacteria of claim 2 to different sub-environments within the same environment.
